(19) United States
(12) Patent Application Publication
(10) Pub. No.: US 2011/0065975 A1
(43) Pub. Date: Mar. 17, 2011

(54) Matrix applicator device and producing method thereof

(75) Inventor: Leonid Kazanskiy, Kowloon (HK)

(73) Assignee: Life Matrix (HK) Ltd.

(21) Appl. No.: 12/584,930

(22) Filed: Sep. 14, 2009

(51) Int. Cl. A61N 2/02; H05K 3/10; H01R 43/00

(52) U.S. Cl. 600/9; 29/846; 29/825

(57) ABSTRACT

A matrix applicator device for modulating biological organism electromagnetic field, includes a base having one or more surfaces, and one or more oscillating circuits for refracting electromagnetic waves of various ranges of the electromagnetic field into coherence form for the biological organism, comprising a plurality of closed loops intersectingly and symmetrically formed on the surface or under the surface of the base, wherein the closed loops are geometrically and/or physically distinguished from the base.

### BACKGROUND OF THE PRESENT INVENTION

1. Field of Invention

The present invention relates to a biological and medical device, and more particularly to a matrix applicator using wave oscillating circuits to improve biological organism functioning.

2. Description of Related Arts

A living cell is a basic structural-functional unit in the human organism. Each cell has a very complex biological structure, where many biochemical reactions occur at the same time and are vital for this cell to exist and perform all of its functions. Any reaction is accompanied by absorption or release of energy in all possible forms, including electromagnetic oscillations. When an electromagnetic signal is registered from a normally functioning cell, then from another one and so on, it turns out that it is unique for each of them. But even in this endless variety it is possible to find something common, inherent in each normally working cell: harmony of the signal.

It should be taken into account that cells in the organism exit only if they are able to interact. This interaction occurs in different forms, at different levels, also at the level of weak electromagnetic interaction. When we register a signal, arising during joint work of a cell population, we can see that the form of this signal is integral and not similar to any of its components. But when the cell population (or the cell system) operates normally, that is it fully carries out the required function with optimum quantitative and qualitative preservation of intrasystem connections, the form of the integral electromagnetic signal of this system will also be harmonious.

It should be particularly emphasized that the detection of the structure of interactions between the parameters of all levels of an organism start from the standpoint of systemic approach, which plays the most important role for understanding the mechanisms of its formation. Disturbance of agreement of functioning between the elements of the functional system, worsening of their synchronization and weakening of connections between them are objective signs of stress and even of exhaustion of the organism's regulatory systems, which is also reflected in the character of an output electromagnetic signal—it ceases to be harmonious.

The cause of the disturbance could be many factors such as un-healthy environment, disease, emotional stimulation, etc. Being exposed under these situations will destroy the harmony of the cell system, and affect the function of the organisms. As a result, the people will feel uncomfortable, pain, or illness. The traditionally treatment for these symptoms is to eliminate the causes of the disturbance, while sometimes it turns out unsuccessfully.

One example is the mobile phone protector. Mobile phone is one of the most widely distributed and comfortable kind of communication in our world. Mobile phone is a small transmitting device. The phone needs to share radio-signals with nearest cellular station for you to hear interlocutor. It is naturally when the station is farther, the phone has to “cry” louder, therefore to increases the power of transmitting signal.

If you clasp the phone to your ear, you thereby put it in immediate proximity to your brain. Even the emission power of mobile phone is many times smaller than the microwave oven which can boil water, the effect of these high frequency electromagnetic waves on human health is highly concerned. The very simply idea to eliminate the effect of the radiation of the mobile phone is to absorb them by any barrier or blocking screen. Most of the protector devices present on the market based exactly on this principle. There is one not the least of the factors: partial absorption of the signal emitted by mobile phone takes inverse effect—the phone starts to emit with greater power. This effect based on the special system that automatically regulates the power of phone signal. Means of that system is to maintain the level of signal connected to the distance between phone and cellular station. When the level of reception decreases that system automatically increases the power of signal radiated by phone. In case of blocking barrier the system reacts same way. As the result the protection from such kind of devices equal to zero. Using of these devices reaches just one real result—decreasing the quality of cellular communication.

In another example, modern people are constantly living under great pressure. Although most of the time people even don't realize it, it will lead to appearance of different neuroses and risen stress. The intensity of nervous system leads to non-controlled muscle stress which evokes problems of spine and joints. Also so on following the chain of consequences. It is not a secret that constant stress is a true cause of the development of different diseases and lowering of the active and valuable life duration. People take different ways to release the pressure. Some activities such as listening music, doing sports, take vacation are some of the best solutions. Or more fundamentally, living in a healthy life style can completely solve the problem. But unfortunately it is really hard for modern people to achieve. Medicine is also being widely used, especially when the symptom is serious. But it is well known that all medicines have side effects. Since human body is an integral organic system, any disturbance, no matter disease or medicine will damage the inner balance, or harmony of the system. Any intension to eliminate one symptom will cause another symptom. So it is necessary to develop a non-invasive method to keep the organism or body in harmony, and recover from outer stimulation, and ultimately keep the human body in health.

### SUMMARY OF THE PRESENT INVENTION

An object of the present invention is to provide a device used for contact and non-contact impact on biological organisms to improve biological organism functioning.

Another object of the present invention is to provide a device to regulate the electromagnetic field of biological organism.

Another object of the present invention is to provide a device to harmonize cell system.

Another object of the present invention is to provide a device to non-invasively heal the disorder of biological organism.

Another object of the present invention is to provide a device which reduces the harmful effects of the environmental radiation.

Another object of the present invention is to provide a device to release pain of particular portion of human body.

Another object of the present invention is to provide a device which doesn't need power supply.

Another object of the present invention is to provide a device which is easy and safe to use.

In order to accomplish the above objects, the present invention provides a matrix applicator device for modulating biological organism electromagnetic field, comprising:

a base having one or more surfaces, and

one or more oscillating circuits for refracting electromagnetic waves of various ranges of the electromagnetic field into coherence form for the biological organism, comprising a plurality of closed loops intersectingly and symmetrically formed on the surface or under the surface of the base, wherein the closed loops are geometrically and/or physically distinguished from the base.

The present invention also provides a method of manufacturing the matrix applicator device for modulating biological organism electromagnetic field, comprising the steps of:

(a) creating a scheme graphic of an oscillating circuit for refracting electromagnetic waves of various ranges into coherence form wherein the oscillating circuit comprises a plurality of closed loops intersectingly and symmetrically arranged; and

(b) generating scheme structures on a base according to the scheme graphic.

The present invention also provides a method of keeping organism in harmonious and health condition by means of the matrix applicator device, comprising steps of:

(a) placing an oscillating circuit in a biological organism electromagnetic field wherein the oscillating circuit comprises a plurality of intersectingly and symmetrically distributed closed loops; and

(b) refracting electromagnetic waves of various ranges of the electromagnetic field into coherence form for the biological organism.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a preferred embodiment of the present invention.

FIGS. 2A to 2B are schematic views of the scheme of the oscillating circuit using rings as basic element according to the above preferred embodiment of the present invention.

FIGS. 3A to 3C are schematic views of the scheme of the oscillating circuit using triangle as basic element according to an alternative mode of the above the preferred embodiment of the present invention.

FIG. 4 is a sectional view of the device using protrusions as the scheme structure according to the above preferred embodiments of the present invention.

FIG. 5 is a sectional view of the device using grooves as the scheme structure according to another alternative mode of the above preferred embodiment of the present invention.

FIG. 6 is a sectional view of the device using impregnated lines as the scheme structure according to another alternative mode of the above preferred embodiment of the present invention.

### DETAILED DESCRIPTION OF THE PREFERRED EMBODIMENT

The present invention uses an innovative approach to keep organism in a harmonious and healthy condition. It is known that all cells, tissues and organs of human organism radiate electromagnetic energy. This radiation can be registered with various diagnostic methods such as electrocardiography (ECG) and electroencephalography (EEG). The infrared radiation of the body can be registered with infrared cameras.

Radiation of each cell is a complex signal which includes the information of the cell's condition. The group of cells as a constituent part of any organ emits synchronized signals. By the shape of ECG signals we can judge about state of cardio-vascular system. The EEG signals carrying information about possible disorders of central nervous system. If a cell is healthy then its emitting pattern will be harmonious, normal. Sick cells show distortion in their behavior of radiation. In harmonious heretofore signal appears chaotic characteristics, signal being distorted. Conclusion: pattern of signal emits by healthy cells carry information about health of the body.

At the same time, all cells, tissues and organs of human organism like of any other biological organism are capable of adaptation. Adaptation is one of the basic phenomena of biology. It is the process whereby an organism becomes better suited to its habitat. Also, the term adaptation may refer to a characteristic which is especially important for an organism's survival. For example, if somebody live in a cold country and then move to the hot climate, firstly this person will experience difficulties to adapt, because the human organism got accustomed to work and live in another conditions. But, after some time had elapsed the tuning for changed conditions is happened and a human begins apprehend hot climate like native. Now if he will be back into a cold country, he will feel cold and all systems of organism need to change them again.

These kinds of changes happen each second on the cell level. If we let the cell for a long time be in the zone of disharmonic, chaotic radiation, then its normal work will be break. If the sick cell places into the field of harmonious, coherent emission, then it will be automatically adapt and the restoration will happen.

To understand the principle of the present invention, it is worth mentioning the phenomenon of oscillating circuit. The common oscillating circuit includes inductor L and condenser (capacitor) C, forming closed electric circuit. In some conditions the circuit can appear self-maintained oscillations. Every oscillating circuit has its own resonance frequency. The frequency is based on two criteria: inductance (L) and capacity of condenser (C). An antenna employs such an oscillating circuit to receive or transfer electromagnetic signals.

The most interesting thing is whatever signal we will have at the input of circuit at the output we will have only signal depended on the L and C parameters. The electric filters based on this feature pass only the signals with appointed frequency and the rest are blocking.

The present invention comprises an oscillating circuit system which is capable of self-excitation activated by the external electromagnetic field. In this case any bio-object such as human, animal or plant located near the device can be the source of such a field. The oscillating circuit system changes the nature of the radiation and reflects it to the bio-object. Due to the adaptation the vita activity parameters of cells tissues and organs are enhanced or recovered.

The basis of the present invention is a passive antenna-filtering complex, made by many closed oscillating circuits and contains information about spectrum of emission of healthy human body's cells.

All scheme made with graphics annular elements intersections which are flat lenses strictly and symmetrically located on/in a plane. These lenses are broadband oscillating circuits act on the basis of human body self-radiation to form reflected radiation packet coordinated by phase, frequency and amplitude. Detailed coordination is ensured by high scheme symmetry and great variety of lens elements achieved due to numerous intersections of basic ring elements.

This lens is an elementary resonator works with electromagnetic waves of definite range.

There are more than half-million such elements on the small surface. Special mutual arranging of lenses give the possibility to filter very wide range of electromagnetic waves—from decimeters' to sub-millimeters'. Most oscillations emitted by human body fall one after the other in this range. Any incoming signal apportions by simple components clear itself from distortions and chaotic additions this way. Thereby the electromagnetic radiation that interacts with device being transformed, the filtering and reflecting already concerted and harmonious signal is happening. Because of the cells' adaptation features, being in the field of such concerted signal start to restore their vital functions if were broken. The cells recover themselves to their native properties. Meanwhile, there is no extraneous and unusual to human body added to the signal, because bio-resonance system just clear and reflect back signals received from the cells.

The present invention provides a graphic scheme—a complicated symmetrical pattern consisting of a large amount of lines with pre-calculated dimensions. This matrix is formed precisely with small particles. Interacting with the emission of cells, this passive resonator stimulates their viability, staring the processes of self-adjustment. In other words, the present invention receives the radiation from the bio-objects, the signals from the cells and due to its special structure harmonizes them.

The present invention performs as a spatial-wave Fourier filter. It allows the resolution of any periodic oscillations into a sum of harmonious components with minimum linear amplitude. The device passively resonates with harmonious components of an electromagnetic signal, which comes from the organism at the point where the device of the present invention is placed and increase it amplitude and strength.

During this process, non-harmonious components of the signal become weaker and may disappear. Therefore, in the device's zone, the conditions of functional activity harmonization are created, which results in the normalization of basic biophysical, biochemical and physiological processes. In other words, owing to a specific fractal-matrix pattern applied on the device “deformed” signals, carrying information about any disturbances in the systemic activity of the organism to the periphery (into biologically active points or zones) “are corrected” at the point of interaction with the present invention and come back, as if “imposing themselves” on the organism, gradually forcing it to reorganize its activity according to the mechanism of biological feedback.

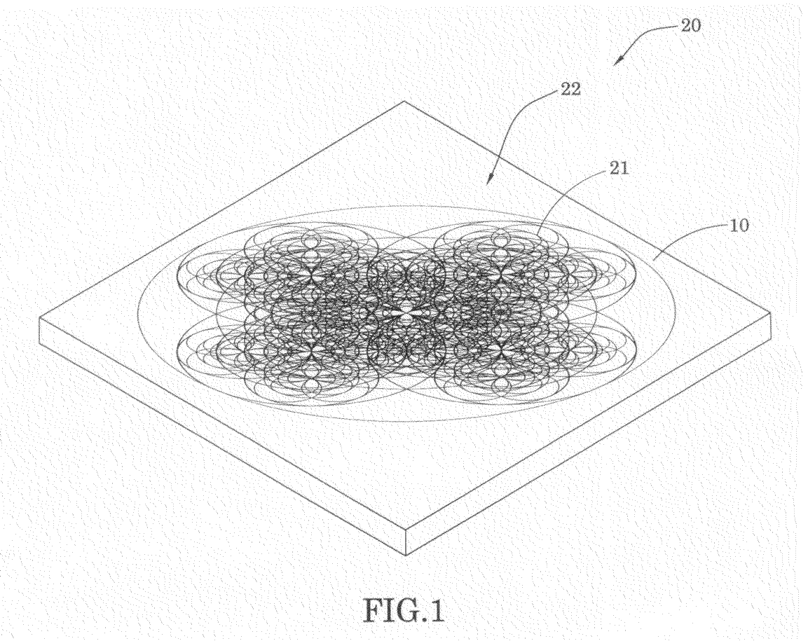

Referring to FIG. 1 of the drawings, the matrix applicator device of the present invention comprises a base 10 having one or more surfaces 11, and a plurality of oscillating circuits 20 formed on the surface or inside of the base 10. The base 10 is a substrate for the oscillating circuits 20 to be formed thereon or therein. It may be in a relatively bulky shape to provide multiple surfaces 11 and considerable volume. Or, it may be just a piece of plate providing one or two surfaces 11 and a predetermined thickness. The size and the shape of the surfaces 11 provided by the base 10 vary according to different applications. For example, the base 10 may be a 14 cm×7 cm square plate, or a circular chip with a 0.5 cm diameter. Many materials can be used to produce the base 10, such as plastic, metal, glass, etc.

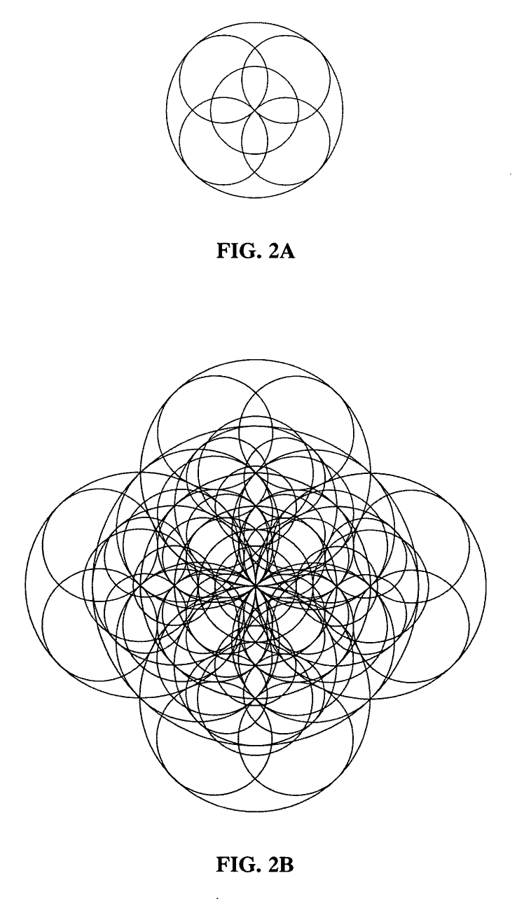

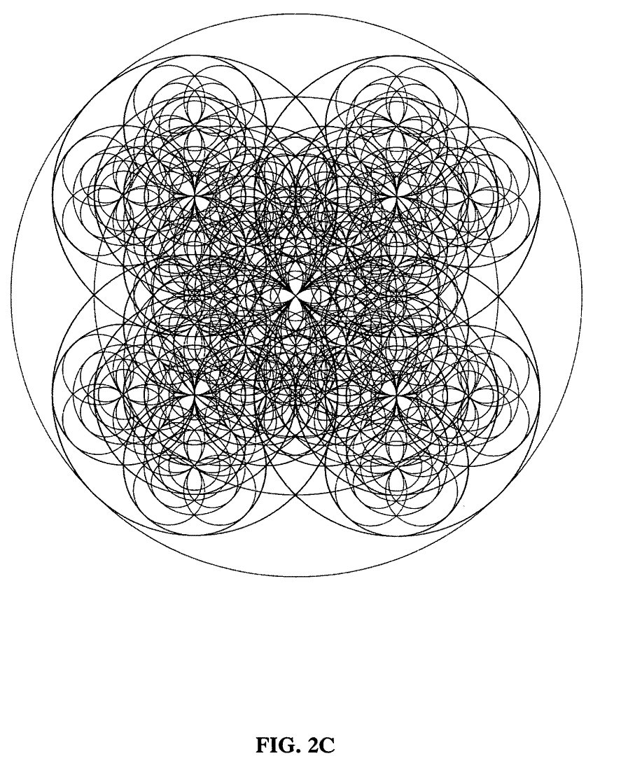

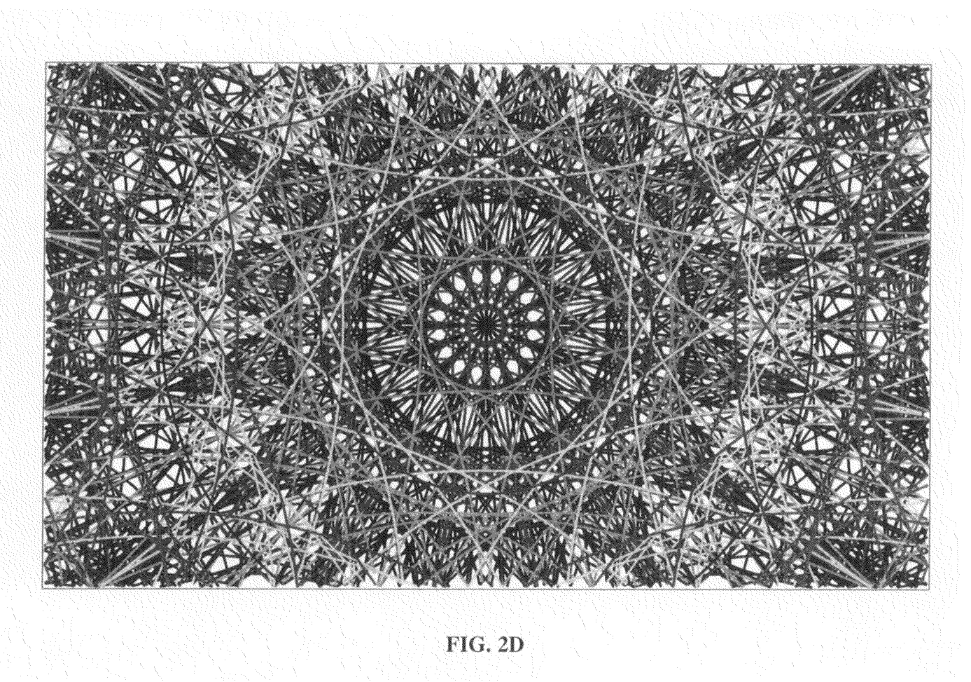

The oscillating circuits 20 are adapted to perform as refraction media of electromagnetic waves of various ranges. Oscillating circuits 20 are passive closed systems wherein the self-excitation effected by electromagnetic self-fields generated by living objects occurs. In order to perform in this way, the oscillating circuits 20 are required to adhere to the maximum achievable level of mutual symmetry of elements. Therefore, the oscillating circuit 20 further comprises a plurality of closed loops 21 arranged symmetrically to form the oscillating circuit 20. Preferably, ring and square elements are employed to form the oscillating circuits 20. Referring to FIG. 2 of the drawings, in a preferred embodiment, the ring elements are utilized to form the oscillating circuits 20. The graphics of the scheme of the oscillating circuits 20 with ring elements is formed in this way:

First, the base ring with radius R is formed. Then 4 points distributed on the boundary of the basic ring are evenly defined. These points are used as ring centers to form 4 rings with radius R which boundaries intercross in the center of the basic ring. Then a concentric ring of the basic ring is formed with radius 2R. Thus the basic scheme is received, as illustrated in FIG. 2A.

Then 4 double-plied copies of the basic scheme are created and are copied with the displacement to 2R radius so that the centers of copies are equidistant. These 5 basic schemes then form a second level scheme.

Then 4 double-pled copies of the second scheme are created and are copied with the displacement to 4R radius so that the centers of the copies are equidistant. The third level scheme is formed, as illustrated in FIG. 2B.

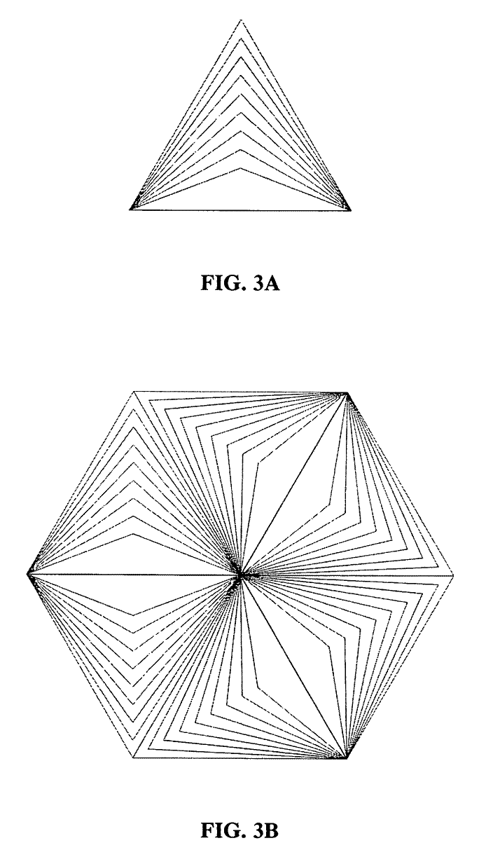

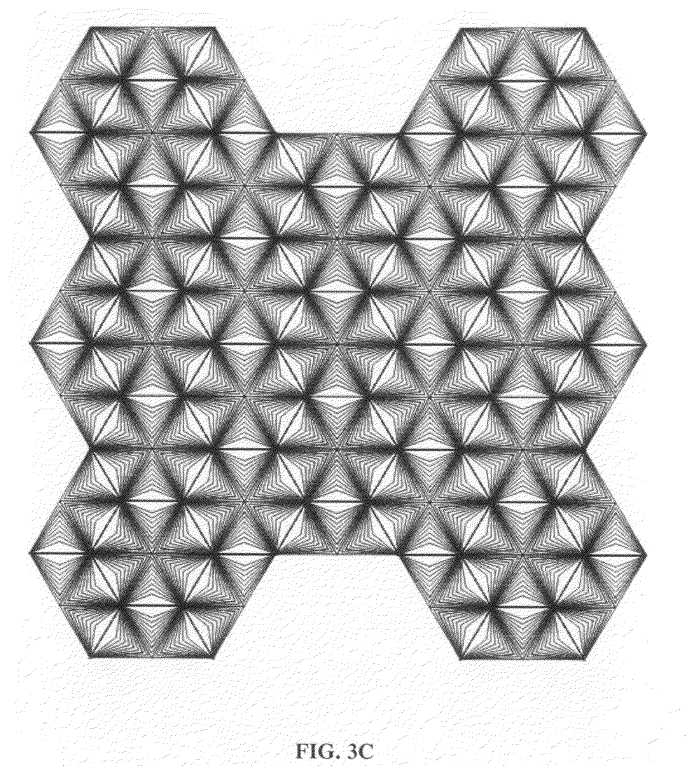

Repeating the process for a certain times, a final scheme is formed as illustrated in FIG. 3C.

The radius R is determined according to the preferred embodiment of the present application. This value may be equal or directly proportional to the existing physical magnitudes such as diameter of hydrogen kernel or biological quantity such as medium interpupillary distance. Besides, to increase the number of elements interacting with different wavelengths copies with R√2 dimension can be used.

Referring to FIG. 3, in an alternative embodiment of the present invention, triangles are used as basic elements. Referring to FIG. 3A, first, an equilateral triangle is formed as the basic triangle. Within the basic triangle a plurality of isosceles triangles sharing the same bottom edge are formed at the same time. As illustrated in FIG. 3B, then 6 copies of the basic triangles are put side by side to form a hexagon. As illustrated in FIG. 3C, more hexagons are copied side by side to create an oscillating circuit 20 scheme.

It is worth mentioning, the oscillating circuits 20 may consist any closed graphics with arbitrary shape.

Once the scheme of the oscillating circuits 20 is defined, there are several methods to realize. The oscillating circuit 20 comprises a plurality of scheme structures 22 formed as the scheme thereof. Preferably, the scheme structure 22 is elongated with a predetermined height and width. The height and width vary from nano meters to micro meters according to different applications and manufacture methods.

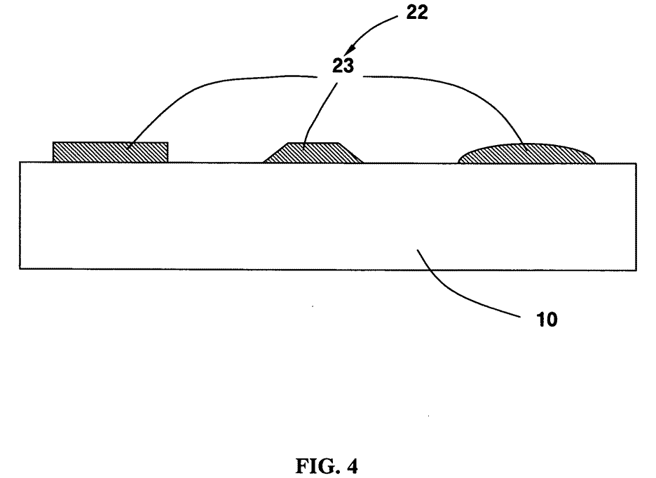

In a preferred embodiment, the scheme structure 22 comprises a plurality of protrusions 23 to form the scheme of the oscillating circuits 20. Referring to FIG. 4, the protrusions 23 extend outwardly from the surface 11 of the base 10. Preferably, the width, height, and profile of the protrusion 23 are uniform on one device, but they may be various on one device either. For different applications, different devices may have the protrusion 23 with different width, height, and profile. It is worth mentioning, the thickness of the base 10 is larger than the height of the protrusions 23.

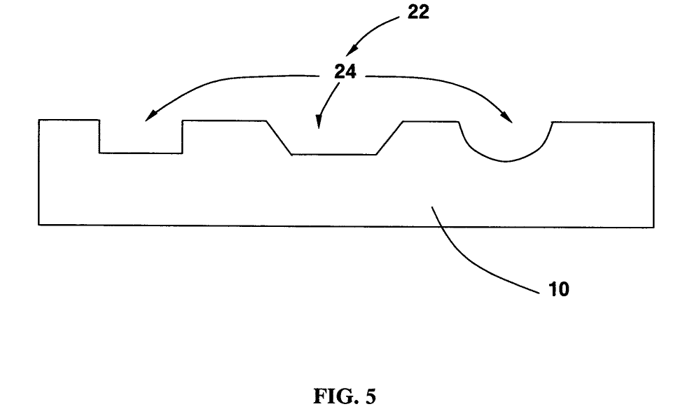

In an alternative embodiment, the scheme structure 22 comprises a plurality of grooves 24 to form the scheme of the oscillating circuits 20. Referring to FIG. 5, the grooves 24 withdraw inwardly from the surface 11 of the base 10. Preferably, the width, depth, and profile of the groove 24 are uniform on one device, but they may be various on one device either. For different applications, different devices may have the grooves 24 with different width, depth, and profile. It is worth mentioning, the thickness of the base 10 is larger than the height of the grooves 24.

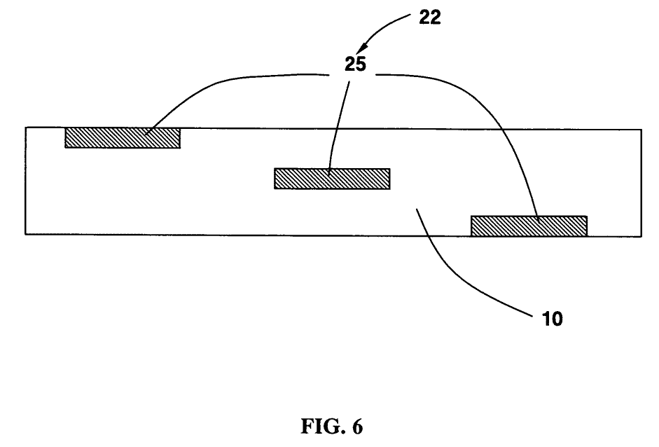

In an alternative embodiment, the scheme structure 22 comprises a plurality of impregnated lines 25 to form the scheme of the oscillating circuits 20. Referring to FIG. 6, the impregnated lines 25 are embedded under the surface 11 of the base 10 or within the base 10. Preferably, the width, height, profile and embedded depth of the impregnated lines 25 are uniform on one device, but they may be various on one device either. For different applications, different devices may have the impregnated lines 25 with different width, height, profile, and embedded depth. It is worth mentioning, the thickness of the base 10 is larger than the height and embedded depth of the impregnated lines 25.

The matrix applicator device of the present invention should be performed as a refraction medium of electromagnetic waves of various which also depends on the material of the base 10 and the oscillating circuit 20. The refraction medium needs to combine more than one material to create the refraction zone. For example, two materials may be used such as silicon and metal, silicon and air, plastic and ink, metal and air etc. The material of the base 10 can be selected from but not limited to various plastic types, ceramics, silicon, precious and semiprecious stones, glass, and various fabric types, etc. The material of the protrusions 23 and impregnated lines 25 may be selected from but not limited to various ink types with or without adding extremely fine metal powders (for example, aluminum, tungsten, molybdenum, silver and other), metallics or metal molecules, any other materials with the help of physical contrast such as color and density contrast for various ranges or frequencies of electromagnetic waves, electrical and thermal conduction contrast or contrast of the density gradient between the materials of the base and/or scheme structures 22 can be created.

The present invention uses several methods to manufacture the matrix applicator device comprising: offset, gravure, screen, flexo/letterpress, chemical etching, deposition of metal particles in vacuum or gas phase or other kinds of deposition, lithography including micro-lithography, ultra-lithography, and nano-lithography, penetration particles of one material into the stratum of another (impregnation), stump extrusion by surface, any kind of pulling (for example, pulling from melt, pulling from flux), laser engraving and other types of engraving by different kind of materials, and any types of crystallization (for example flame fusion, gas-flow).

In general, the method of producing the device for modulating biological organism electromagnetic field comprises the following steps:

1. creating the scheme graphics;

2. creating the mold or photomask;

3. generating scheme structures on the base; and

4. packing the final device.

In step 1, the basic elements and their sizes are determined, and the graphic of the basic elements is generated using image editing programs to generate graphic files, such as CorelDraw, Adobe Illustrator, AutoCAD, etc. Then the graphics of the scheme of the oscillating circuits are generated.

In a preferred embodiment of the present invention, first the base circle with R radius is formed. Then the equidistant points in the quantity multiple of 2 are created on the boundary of the basic ring. These points are assumed as ring centers intercrossing in the center of the basic ring. Thus the basic scheme consisting of minimum 5 (five) ring elements is receive.

Then, the double-plied copy of the basic scheme is created and it is copied with the displacement to 2R radius so that the centers of copies are equidistant. Such double-plied copies are created so many times as it is needed before the major scheme is created. Besides, to increase the number of elements interacting with different wavelengths copies with R√2 and R√3 dimensions can be used.

Then, a defined thickness of the graphics' lines is set to match technological standards. Thus, to make graphics on the plastic base are usually defined by the line width of 25 microns (or less). For making the graphics on crystalline silicon or glass we can define the width of 4-6 microns or less.

The step 2 is to make the mould or mask. Moulds and masks are vary, depends on the bases for making the products.

In step 3, if the scheme structures 22 are protrusions 23, the material of the base is plastic, and the line width is in microns, preferably, printing is used to produce the scheme structures 22. The ink used in printing may be mixed with metal powder. After printing, the ink may be dried and coated with protector or covered by security layers.

If the material of the base is silicon or glass, lithography is preferably utilized to produce the scheme structures 22.

If the scheme structures 22 are grooves 24, etching is preferably utilized to produce the scheme structures 22.

If the scheme structures 22 are impregnated lines 25, deposition if preferably utilized to produce the scheme structures 22.

In step 4, die-cutting and packing are performed.

For different embodiments, the materials and dimensions are various.

In one embodiment, the material of the surface 11 of the base 10 is plastic. The scheme structures 22 are formed by printing from original graphics file. Printing uses inks containing metal powder. The size of the base is can be embodied as 11 cm×3 cm in plate. The width of the protrusions 23 can be embodied as 25 μm±5 μm. Shape and height of protrusions 23 are related to the printing process, usually have half-circle profile. This embodiment is able to be used for pain relief, especially for women during the menstrual period.

In one embodiment, the material of the surface 11 of the base 10 can be made in plastics, fabrics, or leather. Two types of graphics can be used. One is the repeating pattern 15 cm wide printed in rolls. The width of the protrusions 23 can be embodied as 25 μm±5 μm. Second is the repeating pattern printed by silkscreen process on the surfaces 11 of the base 10. The width of the protrusions 23 can be embodied as 50 μm±15 μm.

In one embodiment, chip is used. Preferably, the chip uses circle type of graphics with the diameter of 6.6 mm and 13.2 mm (the diameter of outer circle). Usually chips are manufactured as combination of aluminium graphics performed on silicon plate. Width of the scheme structure can be embodied as 1±0.01 μm (height is equal value), 2±0.01 μm. This value also can vary depends on what result is needed.

Another embodiment is plate of chips which is a combination of at least 4 chips as embodied above arranged as 2×2. The distance between chips should be as the proportion to diameters of circles that whole graphics consisted. Maximum number of chips with 6.6 mm graphics arranged on one plate is 144 (the matrix 12×12 chips). This quantity depends on radius of performed graphics and the radius of silicon wafer. Showed value is based on 200 mm used wafers.

Another embodiment is mobile phone sticker which preferably comprises one chip as embodied above placed in the middle of plastic base having 8-axis graphics performed from aluminium-containing inks or aluminium foil. This combination is protected with plastic cover made by volume-labeling process.

The device of the present invention may have different applications. The main purpose of the device of the present invention is to perform as converters and filters for changing of electromagnetic waves in a very wide range into coherence form.

One of the applications is to act on the living objects. The effect is achieved by placing the device over (amongst) sick cells in their electromagnetic field. The electromagnetic waves pass filtration from distortions (information about disease or dysfunction) and activate self-healing effect of cells (based on possibility of adaptation).

Another application is to act on the liquids, the effect is achieved by placing the device over (in contact, inside, or out of contact, outside) the liquid. That provokes electromagnetic waves phase coherence and filtration them from distortions. The liquid can restore its nature structure owing to that conversion.

Another application is to convert outward emissions. The effect is achieved by filtration and phase coherence of waves getting to the device. Those allot the reflected emission with ability to stimulate of self-healing of biological organisms and their distorted functions.

Another application is to convert light (visible and invisible spectrum). The oscillating circuits placed on transparent surface provoke interference and diffraction effect of the light.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A device for modulating biological organism electromagnetic field, comprising:
a base having at least a surface, and
one or more oscillating circuits for refracting electromagnetic waves of various ranges of said electromagnetic field into coherence form for said biological organism, comprising a plurality of closed loops intersectingly and symmetrically provided at said surface and distinguished from said base.

2. The device, as recited in claim 1, wherein said closed loops are protruding from said surface of said base.

3. The device, as recited in claim 1, wherein said closed loops are withdrawn from said surface of said base.

4. The device, as recited in claim 1, wherein said closed loops are embedded into said base under said surface of said base.

5. The device, as recited in claim 2, wherein a material of said closed loops is different from a material of said base to create a physical contrast between said closed loops and said base.

6. The device, as recited in claim 5, wherein said closed loops are printed with ink on said surface of said base.

7. The device, as recited in claim 6, wherein said ink is added with metal powder.

8. The device, as recited in claim 4, wherein a material of said closed loops is different from a material of said base to create the physical property contrast between said closed loops and said base.

9. The device, as recited in claim 1, wherein said closed loops are triangles.

10. The device, as recited in claim 1, wherein said closed loops are squares.

11. The device, as recited in claim 1, wherein said closed loops are rings.

12. The device, as recited in claim 2, wherein said oscillating circuit comprises:
a basic ring with a radius wherein said radius is equal or proportional to a pre-selected physical magnitude;
a plurality of rings with said radius of said basic ring evenly distributed around said basic ring with a predetermined distance from a center of said basic ring to form a to second level scheme; and
a plurality of copies of said second level scheme symmetrically distributed around said center of said basic ring.

13. The device, as recited in claim 3, wherein said oscillating circuit comprises:
a basic ring with a radius wherein said radius is equal or proportional to a pre-selected physical magnitude;
a number of rings with said radius of said basic ring evenly distributed around said basic ring with a predetermined distance from a center of said basic ring to form a second level scheme;
a plurality of copies of said second level scheme symmetrically distributed around said center of said basic ring.

14. The device, as recited in claim 4, wherein said oscillating circuit comprises:
a basic ring with a radius wherein said radius is equal or proportional to a pre-selected physical magnitude;
a number of rings with said radius of said basic ring evenly distributed around said basic ring with a predetermined distance from a center of said basic ring to form a second level scheme;
a plurality of copies of said second level scheme symmetrically distributed around said center of said basic ring.

15. The device, as recited in claim 2, wherein said oscillating circuits comprises a plurality of basic triangle elements arranged side by side, wherein said basic triangle elements comprises an equilateral triangle, and a plurality of isosceles triangles sharing a same bottom edge of said equilateral triangle therewithin.

16. The device, as recited in claim 3, wherein said oscillating circuits comprises a plurality of basic triangle elements arranged side by side, wherein said basic triangle elements comprises an equilateral triangle, and a plurality of isosceles triangles sharing a same bottom edge of said equilateral triangle therewithin.

17. The device, as recited in claim 4, wherein said oscillating circuits comprises a plurality of basic triangle elements arranged side by side, wherein said basic triangle elements comprises an equilateral triangle, and a plurality of isosceles triangles sharing a same bottom edge of said equilateral triangle therewithin.

18. A method of producing a device for modulating biological organism electromagnetic field, comprising the steps of:
(a) creating a scheme graphic of an oscillating circuit for refracting electromagnetic waves of various ranges into coherence form wherein said oscillating circuit comprises a plurality of closed loops intersectingly and symmetrically arranged; and
(b) generating scheme structures on a base according to said scheme graphic.

19. The method, as recited in claim 18, wherein in the step (a), said closed loops are circles.

20. The method, as recited in claim 19, wherein the step (a) further comprises the steps of:
(a1) creating a basic circle with a predetermined radius;
(a2) creating more than one copies of said basic circle evenly distributed around said basic circle with a predetermined distance from a center of said basic ring to form a second level scheme; and
(a3) creating a plurality of copies of said second level scheme symmetrically distributed around said basic circle to form said scheme graphic of said oscillating circuit.

21. The method, as recited in claim 19, wherein in the step (a), said closed loops are triangles.

22. The method, as recited in claim 21, wherein the step (a) further comprises the steps of:
(a1) creating an equilateral triangle;
(a2) creating a plurality of isosceles triangles within said equilateral triangle sharing a same bottom edge of said equilateral triangle to form a basic triangle element; and
(a3) creating a plurality of copies of said basic triangle element arranged side by side to form said scheme graphic of said oscillating circuit.

23. The method, as recited in claim 19, wherein in the step (a), said closed loops are squares.

24. The method, as recited in claim 18, wherein in the step (b), said scheme structures are formed by depositing materials on one or more surfaces of said base according to said scheme graphic with predetermined dimension, wherein said material is different from the material of said base.

25. The method, as recited in claim 24, wherein in the step (b), said scheme structures are formed by printing with ink.

26. The method, as recited in claim 25, wherein said ink is added with metal powder.

27. The method, as recited in claim 24, wherein in the step (b), said scheme structures are formed by lithography.

28. The method, as recited in claim 18, wherein in the step (b), said scheme structures are formed by making grooves on one or more surfaces of said base according to said scheme graphic with predetermined dimension.

29. The method, as recited in claim 28, wherein in the step (b), said scheme structures are formed by etching.

30. The method, as recited in claim 18, wherein in the step (b), said scheme structures are formed by embedding materials into said base according to said scheme graphic with predetermined dimension, wherein said material is different from the material of said base.

31. A method of keeping organism in harmonious and health condition, comprising the steps of:
(a) placing an oscillating circuit in a biological organism electromagnetic field wherein said oscillating circuit comprises a plurality of intersectingly and symmetrically distributed closed loops; and
(b) refracting electromagnetic waves of various ranges of said electromagnetic field into coherence form for said biological organism.
